(19) United States
(12) Patent Application Publication
(10) Pub. No.: US 2026/0306313 A1
(43) Pub. Date: Oct. 8, 2026

(54) ABSORBENT ARTICLE WITH APERTURED LIQUID ACQUISITION LAYER AND STABILIZING ELEMENTS

(71) Applicants: Vinda Paper (China) Company Ltd, Jiangmen City, Guangdong (CN); Essity Hygiene ang Health Aktiebolag, Göteborg (SE)

(72) Inventors: Huimin WU, Jiangmen City, Guangdong (CN); Yinsheng HUANG, Jiangmen City, Guangdong (CN); Qixing LIU, Jiangmen City, Guangdong (CN); Chengjian LIANG, Jiangmen City, Guangdong (CN); Jinying CHEN, Jiangmen City, Guangdong (CN); Xiao ZHAO, Jiangmen City, Guangdong (CN); Haiying GUO, Jiangmen City, Guangdong (CN); Louise ELIASSON, Göteborg (SE); Magdalena HÖRLE, Göteborg (SE)

(73) Assignees: Vinda Paper (China) Company Ltd, Jiangmen City, Guangdong (CN); Essity Hygiene ang Health Aktiebolag, Göteborg (SE)

(21) Appl. No.: 19/481,631

(22) PCT Filed: Jul. 25, 2023
(86) PCT No.: PCT/EP2023/070570

§ 371 Date: Nov. 5, 2025

(30) Foreign Application Priority Data
May 10, 2023 (CN) 202310527284.9
May 10, 2023 (CN) 202321123630.9

(51) Int. Cl. A61F 13/537; A61F 13/475

(52) U.S. Cl. CPC A61F 13/53743; A61F 13/4756; A61F 2013/53778; A61F 2013/53782

(57) ABSTRACT

The present disclosure relates to a disposable absorbent article having a liquid-permeable topsheet, a liquid-impermeable backsheet, an absorbent core arranged between the topsheet and the backsheet, and a liquid acquisition layer arranged between the topsheet and the absorbent core. The liquid acquisition layer has a length in the longitudinal direction between a front edge and a rear edge, the length having a front portion, a rear portion, and a central portion arranged between the front and rear portions, a width in the transverse direction between first and second edges, and a thickness between a top surface and a bottom surface, the liquid acquisition layer further having a plurality of perforations in the top surface and a plurality of apertures which extend the thickness from the top surface through the bottom surface. Further, the absorbent core has a plurality of stabilizing elements.

### FIELD OF THE INVENTION

The present disclosure relates to disposable absorbent articles, such as sanitary towels, panty liners, incontinence pads or diapers, which are designed to accommodate a balance between user comfort and efficient fluid handling. In particular, these disposable absorbent articles are configured to provide improved fluid handling properties without sacrifice to the fit and are discrete and comfortable to wear.

### BACKGROUND

Disposable absorbent articles, such as sanitary napkins, panty liners, diapers, incontinence pads, and the like need to have good absorptive properties and should provide the wearer with a sense of security against leakage. Such disposable absorbent articles are generally constructed with a liquid permeable topsheet, a liquid impermeable backsheet, and an absorbent structure arranged between the topsheet and the backsheet. A typical topsheet, which is arranged on the user facing side of the absorbent article, may be formed from nonwoven material(s), apertured films, or combinations (e.g., non-woven/film composite or laminate). In contrast, backsheets, which are arranged on the garment facing side of the absorbent articles, are typically made of films, for example, a solid polymer film. In some cases, these films may even include breathable materials that do not impact the imperviousness of the backsheet to fluid leakage. Further, the absorbent cores may be made from a variety of materials, including but not limited to pulp, other fibrous materials, superabsorbent particles or fibers, and combinations.

Depending on the material(s) selected to form the topsheet, backsheet, and absorbent structure, one or more additional layers may be provided between the topsheet and the absorbent structure to further facilitate liquid handling, mainly intake and distribution, into and within the absorbent article. One example of such an additional layer is WO 2005/079542, which describes a three-dimensional, apertured, non-woven material provided in an absorbent article between the topsheet and absorbent core to improve the strikethrough, rewet, and distribution characteristics of the absorbent article. In another example, WO 2019/226827 describes a fluid distribution material for use in an absorbent article that is said to reduce residual wetness; the fluid distribution material has a formed film layer, which includes a plurality of apertured protuberances arranged in a pattern, and a nonwoven layer including a plurality of fibers attached to the formed film layer.

Incorporation of these apertured layers in an absorbent article, however, does not come without disadvantages. One known problem, in particular, with these apertured layers is decreased mechanical strength. The mechanical strength of the layer(s), and by extension, the absorbent article in which the layer is assembled can be decreased due to a reduction in material; because material(s) is removed to create the apertures, which can be rather sizable in dimension, this can create one or more weak spots in the layer(s). And weak spot(s) increase the risk that the material(s) may tear or become otherwise damaged during production and/or assembly.

Despite efforts to improve the fit of and how such articles conform to the body of a wearer during use, there still remains a need for further improvements to enable the article to take a certain shape, distribute absorbed liquid in a desirable and predetermined manner, and prevent leakage, all without decreasing the mechanical strength of the material(s) and/or reducing, if not avoiding the risks of tear(s) or other damages from arising during any of production, assembly, and use of the article.

### SUMMARY OF THE INVENTION

It has been found that despite prior art solutions for disposable absorbent articles, there is still a need to improve the fluid handling properties of such articles, especially with respect to fluid intake and distribution within the absorbent article. In this regard, it is important that the absorbent articles are enabled to spread absorbed fluids in a desired and predetermined manner, and by extension, prevent leakage therefrom. It is of further importance that the fluid handling properties are improved in a manner that does not impinge upon the mechanical stability of the article.

An objective of the present disclosure is thus to provide a disposable absorbent article with improved performance and comfort. Further, it is desirable that the disposable absorbent article is enabled to manage a rapid rush of fluid quickly and efficiently, such as menstrual blood or urine, discharged by the wearer. More particularly, it is desirable that a rapid rush of fluid be able to quickly reach the absorbent member.

It is also an objective to provide a disposable absorbent article with an improved fit in which leakage security is not reduced if not all together unaffected. The disposable absorbent article may be any one of a sanitary towel, a panty liner, an incontinence pad, or a diaper.

It is further desirable to provide a disposable absorbent article that is both discrete and comfortable to wear.

The objectives set forth herein are achieved by the present disposable absorbent article as defined in the appended claims.

Accordingly, in a first aspect of the disclosure, there is provided a disposable absorbent article having a width in a transverse direction (x) along a transverse axis (X) and a length in a longitudinal direction (y) along a longitudinal axis (Y). The absorbent article comprises a liquid-permeable topsheet, a liquid-impermeable backsheet, an absorbent core arranged between the topsheet and the backsheet, and a liquid acquisition layer arranged between the topsheet and the absorbent core. The liquid acquisition layer has a length in the longitudinal direction between a front edge and a rear edge, the length comprising a front portion, a rear portion, and a central portion arranged between the front and rear portions, a width in the transverse direction between first and second edges, and a thickness in the z-direction in between a top surface and a bottom surface, wherein the liquid acquisition layer comprises a plurality of perforations in the top surface and a plurality of apertures, which extend the thickness of the liquid acquisition layer from the top surface through the bottom surface. Additionally, the absorbent core comprises a plurality of stabilizing elements.

The plurality of apertures provided in the liquid acquisition layer facilitate the management of a rapid rush of fluid (e.g., menstrual fluid and/or urine) into the absorbent article by providing a means through which the rush of fluid can rapidly reach the absorbent core. This also contributes to improving the comfort of the absorbent article for a wearer because the fluid can be quickly directed to the absorbent core, reducing the time the fluid spends in the liquid acquisition layer and/or near the topsheet, which in turn, provides an increased sense of dryness for the wearer.

The provision of the plurality of apertures in the liquid acquisition layer, however, also means that there is less material, which can lead to a decrease in mechanical stability of the absorbent article. The absorbent core is provided with a plurality of stabilizing elements to compensate for this decrease.

In certain embodiments, the liquid acquisition layer may have a single-layer structure.

In other embodiments, the liquid acquisition layer may comprise a multi-layered structure. The multi-layered structure may comprise a three-dimensional upper layer and a fibrous sublayer arranged underneath the upper layer. In such embodiments, the three-dimensional upper layer comprises the plurality of perforations. It is not intended for the fibrous sublayer to also comprise the plurality of perforations. The plurality of apertures, however, extend the thickness of the liquid acquisition layer from a top surface of the three-dimensional upper layer through to the bottom surface of the fibrous sublayer.

The plurality of apertures may comprise a first subset arranged in the front portion, a second subset arranged in the central portion, and a third subset arranged in the rear portion.

The first, second, and third subsets may each comprise from 20 to 70%, preferably 20 to 50%, of a total surface area of the respective front, rear and central portions.

The size of individual apertures and/or subsets apertures from the plurality of apertures may be varied. Having apertures of differing sizes can be desirable from a fluid handling standpoint, for example, because some areas of the absorbent article will be expected to receive a relatively larger influx of fluid than other areas during use of the article.

In one non-limiting embodiment, the apertures of at least two of the first, second, and third subsets are equal in size. In another non-limiting example, the apertures of at least two of the first, second, and third subsets are different in size. Additionally or alternatively, the apertures of at least one of the first, second, and third subsets comprise at least two different size apertures.

The apertures have a diameter of from 1 to 5 mm, preferably 1.5 to 3 mm.

The apertures are spaced a distance of from 3 to 30 mm, preferably 5 to 20 mm, apart from the front edge, the first and second edges, and the rear edge of the liquid acquisition layer. By providing a space between the apertures and the edges of the liquid acquisition layer, fluid distribution to the absorbent core can be promoted, particularly in a direction away from the edges of the core, thereby minimizing the risk of leakage. Having the apertures spaced some distance apart from the edges of the liquid acquisition layer may also avoid the formation of a weak spot in the absorbent article, and by extension, reduce the risk that the material(s) will tear or become otherwise damaged during production and/or assembly of the absorbent article.

It should be appreciated that the value(s) and/or the range(s) for the spacing between the apertures and the side edges of the liquid acquisition layer may, in some exemplary embodiments, be correlated with or more suitable for one or more particular types of absorbent article. For example, values and/or subranges from the upper end of the aforementioned ranges may be preferable or more suitable for larger absorbent articles, such as diapers and heavy incontinence products, for which either or both of (i) a relatively larger amount of fluid and/or body exudate intake and distribution and (ii) a relatively higher stress load on the article during use is/are expected. Conversely, values and/or subranges from the lower end of the aforementioned ranges may be preferable or more suitable for smaller absorbent articles, such as small feminine pads and panty liners, for which either or both of (i) a relatively smaller amount of fluid and/or body exudate intake and distribution and (ii) a relatively lower stress load on the article during us is/are expected.

In an exemplary, non-limiting embodiments, the liquid acquisition layer may further comprise a perforated border line around the front, central, and rear portions. The perforated border line is formed of a plurality of discrete holes. The discrete holes also extend the thickness, from the top surface through to the bottom surface, of the liquid acquisition layer. The discrete holes contribute to the fluid handling properties of the absorbent article by providing yet another point of ingress for fluid and/or exudates to quickly reach the absorbent core. In particular, the discrete holes assist with promoting the location and the speed at which incoming fluid is distributed to the absorbent core. Additionally, the perforated border line may serve as an outline of the absorbent core underneath the liquid acquisition layer. As such, the perforated border line may provide the absorbent article with an enhanced visual appearance and/or may signal to the wearer where the portion(s) of the article that are article, and by extension, may contribute to giving the wearer a sense of security for the article against leakage.

The discrete holes are spaced a distance of from 3 to 30 mm, preferably 5 to 20 mm, apart from the front edge, the first and second edges, and the rear edge of the liquid acquisition layer. Having the discrete holes spaced some distance apart from the edges of the liquid acquisition layer is desirable for minimizing the chance that a weak spot is formed in the absorbent article, and by extension, the risk that the liquid acquisition layer will tear or become otherwise damaged during production.

Additionally, it is contemplated that the value(s) and/or the range(s) for the spacing between the discrete holes and the side edges of the liquid acquisition layer may, in some exemplary embodiments, be correlated with and/or more suitable for one or more particular types of absorbent article, for reasons not unlike those discussed above with respect to the apertures. In particular, one or more values and/or subranges from the upper end of the aforementioned ranges may be more preferable for larger absorbent articles, such as diapers and heavy incontinence articles, and conversely, one or more values and/or subranges from the lower end of the aforementioned ranges may be more preferable for smaller absorbent articles, such as small feminine pads and panty liners.

It is further desirable, when the liquid acquisition layer is provided with a perforated border line, that the discrete holes are also spaced some distance apart from each other. In this sense, adjacent holes of the perforated border line may be spaced apart from each other by a distance in a range of from 1 to 8 mm.

The discrete holes have a diameter of from 1 to 5 mm, preferably 1.5 to 3 mm.

It is preferred that the discrete holes of the perforated border line are equal or essentially equal in size to each other. However, the discrete holes may be sized differently with respect to the plurality of apertures. In other words, the discrete holes may be relatively larger or smaller in size than the plurality of apertures. It is further envisioned that the discrete holes may also be equal or essentially equal in size with the plurality of apertures in some embodiments. In any case, the discrete holes may be differently or equally sized with respect to either the entire plurality of apertures or one or more subsets of apertures thereof.

In one exemplary, non-limiting embodiment, the discrete holes may be larger in size than the apertures in one or more of a first subset arranged in the front portion, a second subset arranged in the central portion, and a third subset arranged in the rear portion.

Each of the apertures of the plurality of apertures has a shape. This shape may be the same for all of the apertures. Alternatively, individual apertures and/or subset(s) of apertures may have different shapes. In any case, the shape of the aperture(s) may be selected from one or more of a circle, an oval, a triangle, a square, a diamond, or another polygon.

The apertures may further be provided in a particular arrangement. For example, the apertures may be arranged in rows and/or columns relative to the transverse and longitudinal axes. Providing the apertures in such arrangements can be desirable for controlling fluid handling properties in selected areas of the absorbent articles. These arrangements may also be desirable from a manufacturing standpoint because rows and columns can be easily formed in the liquid acquisition layer and specialized equipment and/or adaptations to the manufacture line, which would/may otherwise be employed to form randomized arrangements, can be avoided. Not to mention, these arrangements further facilitate manufacture, particularly in absorbent articles where the apertures should be synchronized with other elements, such as discrete holes, stabilizing elements and/or shaping lines, with which the absorbent article is provided.

One or more subsets of the plurality of apertures, such as a first subset arranged in the front portion, a second subset arranged in the central portion, and a third subset arranged in the rear portion, can be arranged in respective patterns. The respective patterns may be different in size and/or shape. By having different subsets of apertures with respective patterns, the fluid handling properties of the absorbent article can be desirably tailored, for example, to accommodate for different levels of expected fluid intake in different areas of the absorbent article. The respective patterns may also contribute to comfort and fit of the absorbent article.

To offset a decrease in mechanical stability, the absorbent core is provided with a plurality of stabilizing elements. In an exemplary non-limiting embodiment, the plurality of stabilizing elements comprise a plurality of continuous embossing lines which are arranged in the transverse direction. It is desirable that the continuous embossing lines are oriented to extend in the transverse (cross-direction) of the absorbent article in order to withstand pressure imparted on the absorbent article by the wearer's legs during use.

It is preferable that the plurality of stabilizing elements are spaced a distance from the left and right side edges of the absorbent core. In one non-liming embodiment, the plurality of stabilizing elements are spaced a distance of from 5 to 15 mm, preferably 5 to 10 mm, apart from left and right side edges of the absorbent core. Spacing the stabilizing elements a distance apart from the side edge of the absorbent core is desirable for reducing the risk that side leakage will occur.

The absorbent core has a length in the longitudinal direction (y) between a front transverse edge and a rear transverse edge. The length may comprise a front section, a rear section, and a central section arranged between the front and rear sections. The absorbent core also has a width in the transverse direction (x) between left and right side edges.

One or more of the front, central and rear sections of the absorbent core can comprise a subset of the plurality of stabilizing elements. For example, in one non-limiting embodiment, the plurality of stabilizing elements comprises a first subset arranged in the front section of the absorbent core, a second subset arranged in the central section, and a third subset arranged in the rear section.

It is preferable that the stabilizing elements are arranged so as to not overlap with the plurality of apertures. It is also preferable that the stabilizing elements are arranged to not overlap with the perforated border line that may be provided around the front, central and rear portions of the liquid acquisition layer. Overlap between the stabilizing elements and the plurality of apertures and/or the plurality of discrete holes of the perforated border line of the liquid acquisition layer are undesirable because the overlap can create space between the topsheet and the absorbent core in the assembled absorbent article. This means that it becomes difficult, if not impossible, for the topsheet to be in direct contact with or touch the absorbent core.

It is further preferable that the stabilizing elements are arranged between adjacent rows of apertures. Again, the plurality of apertures may comprise one or more subsets and the stabilizing elements may be arranged between the adjacent rows of apertures in one ore more of these subsets. In one non-limiting example, the plurality of stabilizing elements are arranged between adjacent rows of apertures in one or more of a first subset arranged in a first portion, a second subset arranged in the central portion, and a third subset arranged in the rear portion.

The absorbent core may further comprise a first pair of continuous shaping lines arranged between the front section and central section and a second pair of continuous shaping lines arranged between the central section and the rear section. A central rear shaping line may also be arranged along the longitudinal axis (Y) in a center of the rear section. These shaping lines promote preferential bending of the article during use and thus the fit of the article can be improved in both the front and the rear of the article.

In an exemplary, non-limiting embodiment, the first pair of continuous shaping lines comprise a left front line and a right front line. Both the left front line and right front line originate from a respective side edge of the absorbent core and extend upward at an angle toward a central longitudinal axis of the absorbent core. Further, it is preferable that the left front line originates from the left side edge in the central section of the absorbent core and the right front line originates from the right side edge in the central section of the absorbent core. The continuous shaping lines may, therefore, be understood to be arranged in y-direction of the absorbent article.

In an exemplary non-limiting embodiment, the second pair of continuous shaping lines comprise a left rear line and right rear line. Both the left rear line and right rear line originate from a respective side edge of the absorbent core and extend downward at an angle toward a central longitudinal axis of the absorbent core. Further, it is preferable that the left rear line originates from the left side edge in the central section of the absorbent core and right rear line originates from the right side edge in the central section of the absorbent core. The continuous shaping lines may, therefore, be understood to be arranged in y-direction of the absorbent article.

Any one or more of the first pair of continuous shaping lines, the second pair of continuous shaping lines, and the central rear shaping line may be essentially straight lines produced by means of a groove or line compression tool having a shape of a straight line. However, due to the nature of compressible materials used in the absorbent articles minor variations in the absorbent article may occur. It is further envisioned that the shaping lines described herein may also constitute lines in which material has been removed.

By providing continuous shaping lines as described above, folding of the article is promoted in a manner that more closely approximates the anatomy of the wearer, especially in the front and central portions of the absorbent article. The first pair of continuous shaping lines especially promote deformation which conforms to the anatomy of the wearer in an improved manner because these lines enhance the bending of the front portion of the article towards the pubic area of the wearer.

The liquid acquisition layer and the absorbent core may have different dimensions. In one non-limiting example, the length and the width of the liquid acquisition layer are larger than the respective length and the width of the absorbent core. Accordingly, when assembled, the liquid acquisition layer overlays the entire absorbent core.

The liquid acquisition layer can take the form of a number of different shapes. For example, in one non-limiting embodiment, the liquid acquisition layer can have a rectangular shape. This is particularly desirable from a manufacturing standpoint because a rectangle is a relatively simple shape that well accommodates processing through a manufacturing line and requires minimal, if no cuts, to shape the material. This also means the material waste is minimized, if not all together avoided.

In another non-limiting embodiment, the liquid acquisition layer may have a shape comprising a head part in the front portion, first and second leg members that extend symmetrically about a central longitudinal axis in the central and rear portions, and a transition part arranged between the head part and the first and second leg members, which defines a minimum width in the transverse direction. This particular shape, with a head portion, transition part, and first and second leg members, facilitates bending of the absorbent article in particular areas during use. This flexibility of the absorbent article thus can be understood to improve the fit of the absorbent article, and by extension, the comfort level of the wearer.

The absorbent core may be formed from a single material or a combination of materials. In particular, the absorbent core may comprise one or more of pulp, natural fibers, synthetic fibers, tissue, superabsorbent polymers, absorbent foam materials, absorbent nonwoven materials, and the like.

It is also envisioned that the absorbent core can be formed from one or more absorbent members. In one non-limiting example, the absorbent core may comprise a first absorbent member and a second absorbent member. Further, the first and second absorbent members may be formed of the same, similar, or different materials, which can be readily selected by one or skill in the art, depending on the desired absorbency properties. In one non-limiting example, the first absorbent member may comprise a nonwoven fabric structure and the second absorbent may comprise a mixture of pulp fibers and a superabsorbent material.

Additionally or alternatively, an absorbent member formed from a mixture of fibers and superabsorbent material can also be structured to form a gradient. In this sense, the mixture of pulp fibers and superabsorbent polymer can be arranged such that a proportion of the pulp fibers gradually increases from a top surface of the absorbent member to the bottom surface of the absorbent member.

The disposable absorbent article may also be provided with one or more colors. Incorporating visibly different colors can assist to enhance the visual appearance of the absorbent article and/or provide the wearer with an indication as to how the absorbent article should be oriented during use. In one non-limiting example, the liquid acquisition layer may comprise a first color, the absorbent core comprises a second color that is different from the first color, and the topsheet is partially or fully transparent.

Further objectives, features and advantages of the present disposable hygiene article are described in the detailed description below with reference to the appended drawings.

### BRIEF DESCRIPTION OF THE DRAWINGS

The present invention will be discussed in more detail below, with reference to the attached drawings, in which:

FIG. 1 shows an exploded view of an exemplary disposable absorbent article according to the present disclosure;

FIG. 2 shows an embodiment of a disposable absorbent article according to the present disclosure;

FIG. 3 shows an embodiment of a disposable absorbent article according to the present disclosure with patterned subsets of apertures;

FIG. 4 shows an embodiment of a disposable absorbent article according to the present disclosure with a perforated border line;

FIG. 5 shows an embodiment of a disposable absorbent article according to the present disclosure with stabilizing elements;

FIGS. 6A and 6B each show a cross-section of an embodiment of the absorbent core taken along the line VI of FIG. 5;

FIG. 7 shows an embodiment of a disposable absorbent article according to the present disclosure with stabilizing elements and shaping lines; and

FIGS. 8A and 8B show an exemplary liquid acquisition layer.

### DETAILED DESCRIPTION

The present disposable absorbent article is an absorbent article aimed for personal hygiene and may be, for example, a sanitary towel, a panty liner, an incontinence pad or diaper. Such articles are commonly used for acquisition and storage of bodily exudates such as urine, faeces, and menstrual fluid. The absorbent article is disposable, which means that it is intended for a single use and disposed thereafter, instead of being cleaned and reused.

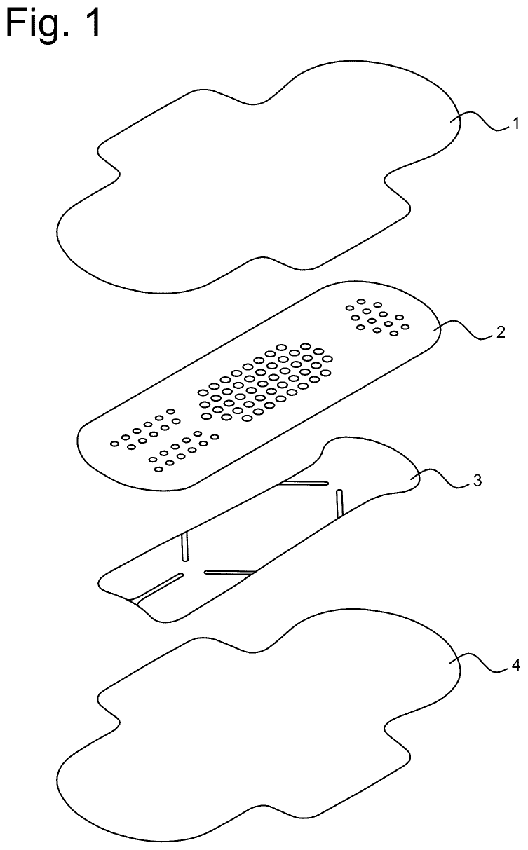

An exemplary disposable absorbent article in accordance with the present disclosure is show in an exploded view in FIG. 1, in the exemplary form of a sanitary napkin. It should be understood that even though the disposable absorbent article is described hereafter as a sanitary napkin, this form is merely exemplary, and it is contemplated that the disposable absorbent article may be provided in other forms, such as a panty liner, an incontinence pad, and a diaper, and as otherwise described or referenced herein.

As shown in FIG. 1, the absorbent article generally comprises four components: a liquid permeable topsheet 1, a liquid acquisition layer 2, an absorbent core 3, and a liquid-impermeable backsheet 4. Structurally, the liquid acquisition layer and the absorbent core are arranged between the topsheet and the backsheet, with the liquid acquisition layer arranged adjacent to the topsheet and the absorbent core arranged adjacent to the backsheet.

The topsheet lies in direct contact with the wearer's body and should therefore be soft and comfortable. The topsheet can comprise a nonwoven material, e.g. spunbond, meltblown, carded, hydroentangled, wetlaid etc. Suitable nonwoven materials can be composed of natural fibers, such as wood pulp or cotton fibers, man-made fibers, such as polyester, polyethylene, polypropylene, viscose, or from a mixture of natural and man-made fibers. Apertured plastic films may also be used as topsheet materials. In any case, suitable topsheet materials should be soft and non-irritating to the skin and be readily penetrable by bodily fluids, e.g. urine or menstrual fluid.

The backsheet lies in contact with the wearer's garments. The backsheet refers to the liquid impervious material forming the outer cover of the hygiene article. The backsheet can comprise a thin plastic film, e.g. a polyethylene or polypropylene film, a nonwoven material coated with a liquid impervious material, a hydrophobic nonwoven material, which resists liquid penetration, or a laminate of a plastic film and a nonwoven material. Other laminate materials which are suitable for use as the backsheet are laminates of a nonwoven material and high loft material. The backsheet material may be breathable so as to allow vapor to escape from the absorbent core, while still preventing liquids from passing there through. Examples of breathable backsheet materials are porous polymeric films, nonwoven laminates of spunbond and meltblown layers and laminates of porous polymeric films and nonwoven materials. Preferably, the backsheet comprises nonwoven material in at least the garment-facing surface thereof.

The liquid acquisition layer is located between the topsheet and the absorbent core and may suitably be placed on top of the absorbent core. The liquid acquisition layer is adapted to quickly receive and temporarily store discharged liquid before it is absorbed by the absorbent core. Such acquisition/distribution layers may be composed of, for example, airlaid nonwoven, spunlace nonwoven, high loft nonwoven, foam materials, and/or combinations thereof. Further, the nonwoven material may be hydrophilic and/or a hydrophilic material may be obtained by adding surfactant.

The liquid acquisition layer can be formed of a single layer. Alternatively, the liquid acquisition layer can have a multi-layered structure. When provided as a multi-layered structure, the liquid acquisition layer may comprise a three-dimensional upper layer and a fibrous sublayer arranged underneath the upper layer.

The absorbent core is the absorbent structure of the article which acquires and stores bodily fluids and exudates. The absorbent core may be of any conventional kind. Examples of commonly occurring absorbent materials are cellulosic fluff pulp, tissue, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fiber, flake, sphere, etc. possesses a higher crosslink density than the inner portion of the superabsorbent. The superabsorbent materials may be in any form suitable for use in absorbent composites including particles, fibers, flakes, spheres, and the like. A high absorption capacity is provided by the use of high amounts of superabsorbent material. Thin absorbent cores, which are common in for example baby diapers and incontinence guards, often comprise a compressed, mixed or layered structure of cellulosic fluff pulp and superabsorbent polymers. The size and absorbent capacity of the absorbent core may thus be varied to suit different product types, such as sanitary napkins for adult incontinent persons or panty liners.

For embodiments according to the present disclosure in which the absorbent core comprises pulp, wood-based or otherwise, and similarly for embodiments in which the absorbent core comprises both pulp and a superabsorbent polymer(s) (SAP), it is preferable that the total basis weight is in a range of from 250 to 400 gsm. It is also preferable that such absorbent cores have a density in a range of from 95 to 160 kg/m³.

In certain embodiments, the absorbent core can be of unitary construction, whereby for example the manufacturing process can be simplified. The phrase “unitary construction” in the present context is intended to mean that the absorbent core is constructed from essentially one type of material, this being essentially the same material, or essentially the same combination of two or more materials throughout the absorbent core. Variations in density and concentration of the material may occur, but these are limited to those which may be obtained without incorporation of regions which have been formed separately and then physically joined to each other. For example, when the absorbent core comprises a matrix of hydrophilic fibers and superabsorbent material as described above, the relative concentrations of superabsorbent material and fibers may be different in different parts of the core. However, the absorbent core of unitary construction does not comprise layers or laminates of different composition. Likewise, variations in the density or concentration of various components across the longitudinal direction, the transverse direction or the thickness direction of the absorbent core are acceptable, yet the core should not comprise areas or layers of different composition which are formed separately and later joined together.

In certain embodiments, the absorbent core can comprise a plurality of absorbent members. For example, in one non-limiting embodiment, the absorbent core comprises a first absorbent member and a second absorbent member. In the assembled absorbent article, the first absorbent member is arranged adjacent to the liquid acquisition layer and the second absorbent is arranged underneath the first absorbent member and adjacent to the backsheet. The first absorbent member may comprise a nonwoven fabric substrate. The second absorbent member may comprise a mixture of pulp fibers, preferably wood pulp fibers, and a superabsorbent material. In such embodiments, it is further envisioned that the blend of pulp fibers and superabsorbent material optionally form a gradient in which a proportion of the pulp fibers generally increases from a top surface of the absorbent member to the bottom surface of the absorbent of member. Stated differently, in an assembled absorbent article, the proportion of pulp fibers in the absorbent member proximal to the backsheet should be relatively higher than the proportion of pulp fibers in the absorbent member proximal to the liquid acquisition layer.

Additionally, one or more of individual components of disposable absorbent article, i.e., the topsheet, the liquid acquisition layer, the absorbent core, and the backsheet may also be provided with one or more colors to enhance the visual appearance of the absorbent article and/or provide the wearer with an indication as to how the absorbent article should be oriented during use. In one non-limiting example, the liquid acquisition layer may comprise a first color, the absorbent core comprises a second color that is different from the first color, and the topsheet is partially or fully transparent.

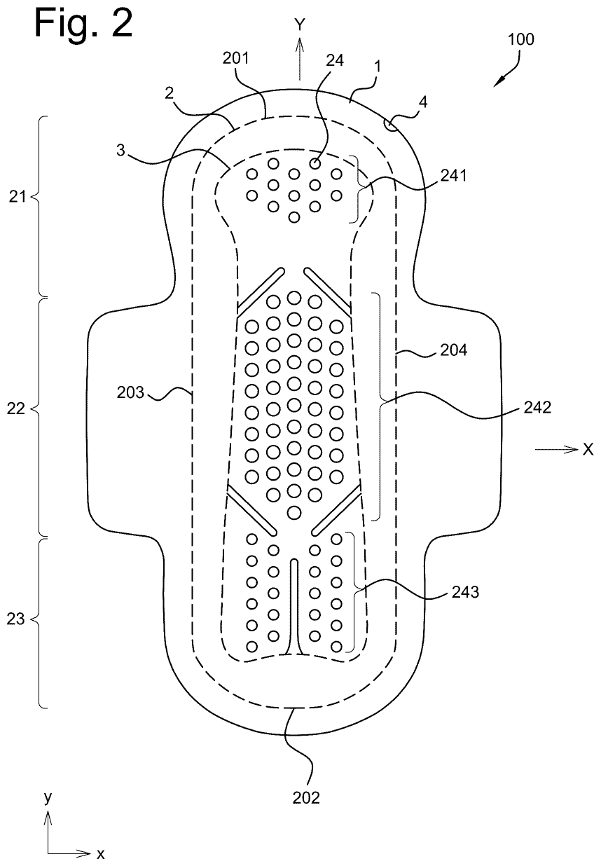

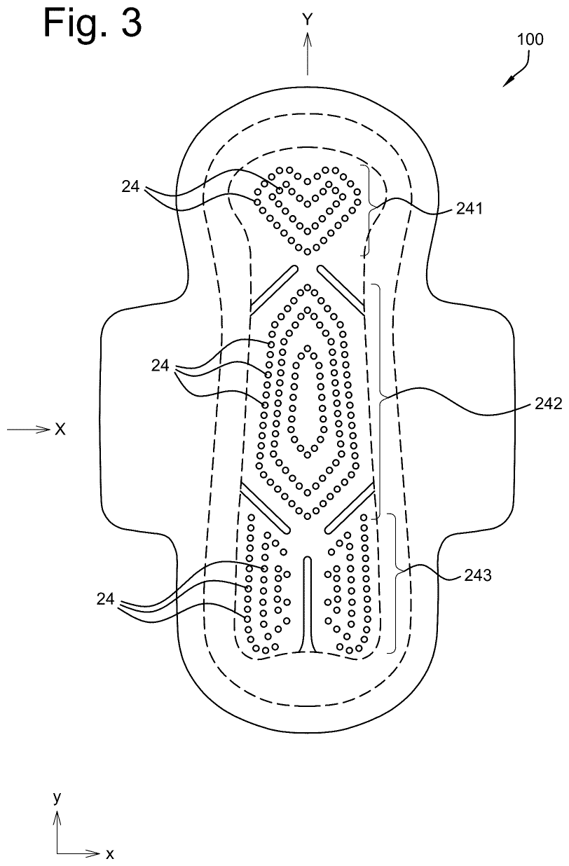

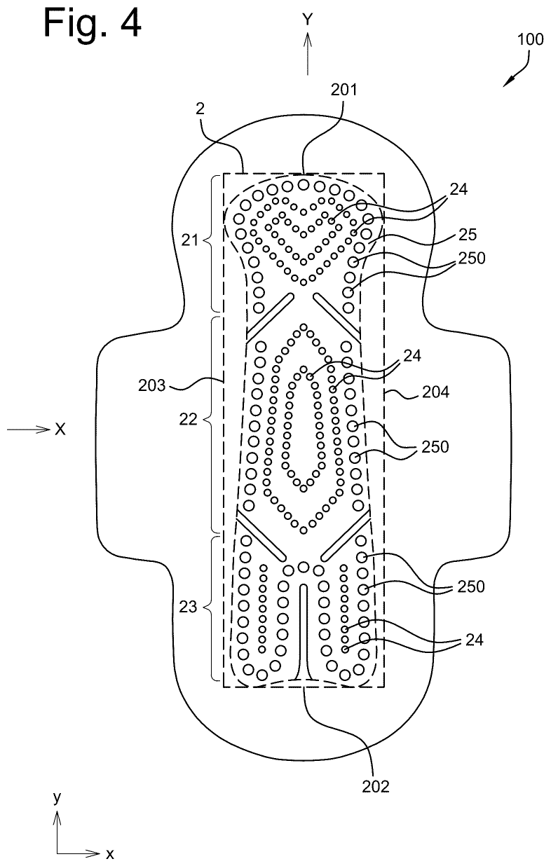

An embodiment of the disposable absorbent article is shown in plan view in FIG. 2. In general, the absorbent article 100 has a width in the transverse direction (x) along a central transverse axis (X) and a length in the longitudinal direction (y) along a central longitudinal axis (Y). The liquid acquisition layer 2 is arranged between the topsheet 1 and the absorbent core 3. The liquid acquisition layer has a length in the longitudinal direction (y) between a front edge 201 and a rear edge 202 and a width in the transverse direction (x) between first and second edges 203, 204. Along the length, the liquid acquisition layer is divided into a front portion 21, a rear portion 23, and a central portion 22 arranged between the front and rear portions. The front, central and rear portions are discrete portions that do not overlap with one another and together may account for the entire longitudinal length of the liquid acquisition layer. Also, the liquid acquisition layer may have a generally rectangular shape with rounded edges at the front and rear edges, as shown in FIG. 2. However, it is to be understood that this generally rectangular shape is merely exemplary and the the liquid acquisition layer can take the form of other shapes, as desired, and as readily selected by one of skill in the art. Exemplary liquid acquisition layers having other shapes are shown for example, in FIGS. 3 and 4. FIG. 4, in particular, depicts a rectangular liquid acquisition layer, which as indicated above, is desirable for manufacturing purposes since waste material can be reduced, if not all together avoided.

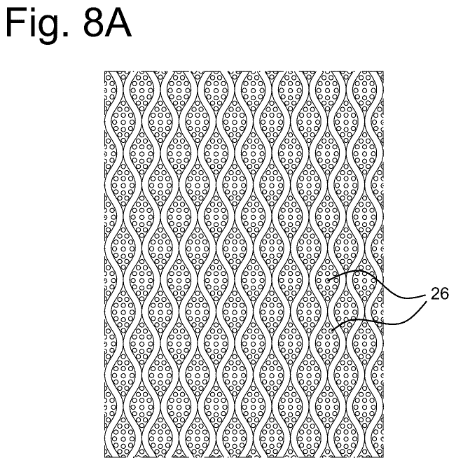

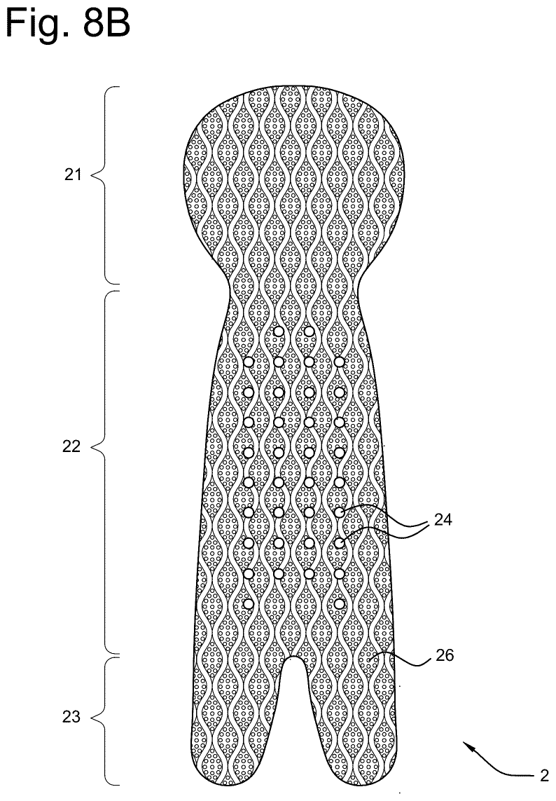

In another non-limiting embodiment, the liquid acquisition layer may have a shape comprising a head part in the front portion, first and second leg members that extend symmetrically about the central longitudinal axis in the central and rear portions, and a transition part arranged between the head part and the first and second leg members. The transition part defines a minimum width in the transverse direction. Preferential bending may be imparted to the absorbent article by way of this particular shape. A non-limiting exemplary liquid acquisition layer having this shape is depicted in FIG. 8B.

In any case, the liquid acquisition layer is dimensioned such that the length and width are smaller than the respective lengths in the longitudinal direction and the respective widths in the transverse direction of the topsheet and the backsheet. Further, the liquid acquisition layer may be dimensioned such that the length and width are larger than the respective length in the longitudinal direction and the respective width in the transverse direction of the absorbent core. By way of this arrangement, the liquid acquisition layer may overlay the entire absorbent core, when assembled, in an absorbent article.

The liquid acquisition layer is provided with a plurality of perforations 26 in the top surface adjacent to the topsheet in an assembled article. This is shown, for example, in FIG. 8A. These perforations may be formed by puncturing the material or by another alternative manner by one of skill in the art. The perforations contribute to the fluid handling properties by facilitating the direction of fluid flow to the absorbent structure below. Additionally, the perforations may, in some embodiments, have a funnel shape, which may be particularly desirable for channeling fluid and/or exudates toward the absorbent core.

The liquid acquisition layer is further provided with a plurality of apertures 24 that extend through the entire thickness, from a top surface through the bottom surface, of the liquid acquisition layer. The plurality of apertures may comprise one or more subsets of apertures. FIG. 2, for example, depicts an article with three subsets of apertures. A first subset 241 is arranged in the front portion, a second subset 242 is arranged in the central portion, and a third subset 243 is arranged in the rear portion.

The subsets of apertures can have a variety of configurations. Depending on how they are arranged, the subsets can impact the fluid handling properties, structural stability, and/or the fit of the absorbent article. Factors that may be taken into consideration when selecting a configuration include, but are not limited to, size, shape, number, position and combinations thereof.

Each subset of apertures also comprises a percentage of the total surface area of the respective section of the liquid acquisition layer in which the subset(s) is/are positioned. It is also envisioned that the surface area percentage of a subset of apertures may also be defined in terms relative to the overall liquid acquisition layer, rather than a portion thereof. In one non-limiting example, the first, second, and third subsets each comprise from 20 to 70%, preferably 20 to 50%, of a total surface area of the respective front, central, and rear portions.

In terms of size, each aperture may have a diameter of from 1 to 5 mm, preferably 1.5 to 3 mm.

In terms of shape, each aperture may have a shape selected from one or more of a circle, an oval, a triangle, a square, a rectangle, a diamond, or other polygon. It is to be understood that these shapes are merely exemplary and that the skilled person could readily select any other suitable shape not as desired.

Turning back to the non-limiting, exemplary absorbent article of FIG. 2, each of the first, second and third subsets have a different configuration. Relative to the other two subsets, the first subset has the fewest number of apertures. In this example, the apertures of the first subset are divided across five columns, each of which contain either 2 or 3 individual apertures, and are staggered at different positions in the longitudinal direction. Together the columns form a heart-like shape arranged such that it is symmetric about the central longitudinal axis (Y) of the absorbent article.

The second subset of apertures in FIG. 2 has the largest number of apertures. As the central portion of the absorbent article is typically expected to receive the greatest influx of fluid and/or exudates, it is desirable for the second subset arranged in this area to contain to greatest number of apertures to receive and direct this flow of fluid and/or exudates into the absorbent core below. Like the first subset, the apertures are also divided across five columns, which contain varying numbers of individual apertures. The columns are staggered at different positions in the longitudinal direction but are arranged so as to be symmetric about the central longitudinal axis of the absorbent article. Further, all of the apertures of second subset are equally or at least essentially equally sized within the subset, and are relatively larger than the apertures of the first and third subsets.

The third subset has more apertures than the first subset and less than the second subset. In this non-limiting example, the apertures of the first and third subsets are equally or at least essentially equally sized with each other. The apertures are divided across four columns, each of which contain either 5 or 6 individual apertures. Again, the columns are staggered at different positions in the longitudinal direction but are arranged so as to be symmetric about the central longitudinal axis. This symmetry present in all three subsets contributes to how the article is intended to be oriented during use, may enhance the visual appearance of the article, and also contributes to the overall fit of the article for the wearer. More specifically, the subsets may function as visual cues for the wearer as to the intended folding and shaping of the absorbent article, thereby indicating how the article should be placed in the wearer's undergarment. The subsets may further provide the absorbent article with a slight pre-shaping, which contributes to an improved fit.

Also, any one or more of the first, second, and third subsets may be positioned in their respective portions of the liquid acquisition layer such that the apertures fully overlay the absorbent core in the assembled article. Having the apertures fully overlay the absorbent core below in the assembled article contributes to improved fluid handling characteristics since the fluid flow may be immediately directed to the absorbent core via the apertures and may contribute to reducing the risk of leakage, from the sides or otherwise. It is further desirable that the apertures are spaced a distance apart from the front edge, the first and second edges, and the rear edge of the liquid acquisition layer to reduce, if not avoid, the risk of side leakage. In non-limiting embodiments of the present disclosure, the distance at which the apertures are spaced apart from the edges of the liquid acquisition layer may range from 3 to 30 mm, and preferably from 5 to 20 mm.

FIG. 3 shows a plan view of another embodiment of the disposable absorbent article according to the present disclosure and similar to the exemplary absorbent article shown in FIG. 2. The liquid acquisition layer of this absorbent article is also provided with three subsets of apertures—a first subset 241 in the front portion, a second subset 242 in the central portion, and a third subset 243 in the rear portion. In this non-limiting example, the apertures of all three subsets are equally or essentially equally sized with each other. Further, the apertures are arranged in respective patterns that are different, but each respective pattern is arranged as to be symmetric about the central longitudinal axis. In the first subset, the apertures are provided in a heart-like shape pattern that includes a smaller heart-like shape of apertures surrounded by and spaced a distance apart from a larger heart-like shape of apertures.

The apertures of the second subset are arranged in a pattern of three closed loops, each of which, in this non-limiting example, has a hexagon-like shape. A first inner loop is arranged in the center of the central region and is surrounded by and spaced a distance apart from a second intermediate loop. The second intermediate loop is further surrounded by and spaced a distance apart from a third outer loop. Along the central longitudinal axis (Y) of the absorbent article, the section of each loop that is oriented most proximal to the front portion has an inverted V-shape, and conversely, the section of each loop that is oriented most proximal to the rear potion has a V-shape.

The apertures of the third subset are arranged in yet another respective pattern that is different from the respective patterns of both the first and second subsets of this exemplary absorbent article. In the third subset, the apertures are generally arranged in a number of a columns that is equal on both sides of the central longitudinal axis. The columns have different numbers of individual apertures and are staggered at different positions in the longitudinal direction but are arranged so as to be symmetric about the central longitudinal axis (Y) of the absorbent article. By way of the staggering of the columns, the first aperture in each column closest to the central portion may form an imaginary line that extends downward at an angle toward the central longitudinal axis. Similarly, the last or penultimate aperture in each column may form an imaginary line that extends upward at an angle toward the central longitudinal axis.

As shown in FIG. 4, the liquid acquisition layer may also be provided with a perforated border line 25 that is formed of a plurality of discrete holes 250. Like the plurality of apertures, the discrete holes extend the thickness of the liquid acquisition layer from the top surface through the bottom surface. The perforated border line is preferably provided around the front, central, and rear portions and separates the plurality of apertures from the front and rear edges in the longitudinal direction and the first and second edges in the transverse direction. The discrete holes and the apertures should be suitably spaced from each other to avoid overlap as this could introduce weak spots into the liquid acquisition layer, which could in turn lead to tears and/or otherwise damage the liquid acquisition layer. It is further preferable that the perforated border line is generally continuous around the front, central, and rear portions.

Adjacent holes of the perforated border line are spaced a distance apart from each other and should not overlap. The distance between adjacent holes may be a distance in a range of from 1 to 8 mm.

In an exemplary, non-limiting embodiment, the discrete holes may have a diameter of from 1 to 5 mm, preferably 1.5 to 3 mm.

Additionally, the discrete holes are spaced a distance apart from the front edge, the first and second edge, and the rear edge of the liquid acquisition layer to reduce, if not avoid, the risk of side leakage. In non-limiting embodiments of the present disclosure, the distance at which the apertures are spaced apart from the edges of the liquid acquisition layer may range from 3 to 30 mm, and preferably from 5 to 20 mm.

The perforated border line may also have a shape similar to that of the absorbent core. This can provide definition to the absorbent core arranged below the liquid acquisition layer. It may also enhance the visual appearance of the absorbent core to the wearer, which can by extension, provide the wearer with an increased sense of security of the article against leakage.

In some embodiments, the absorbent core may be provided with one or more shaping lines, as discussed in further detail hereafter. In such embodiments, it is further preferable that the discrete holes of the perforated border line are suitably spaced a distance apart from the shaping line(s). Overlap between the discrete holes and the shaping lines may weaken the mechanical stability of the absorbent article and thus should be avoided.

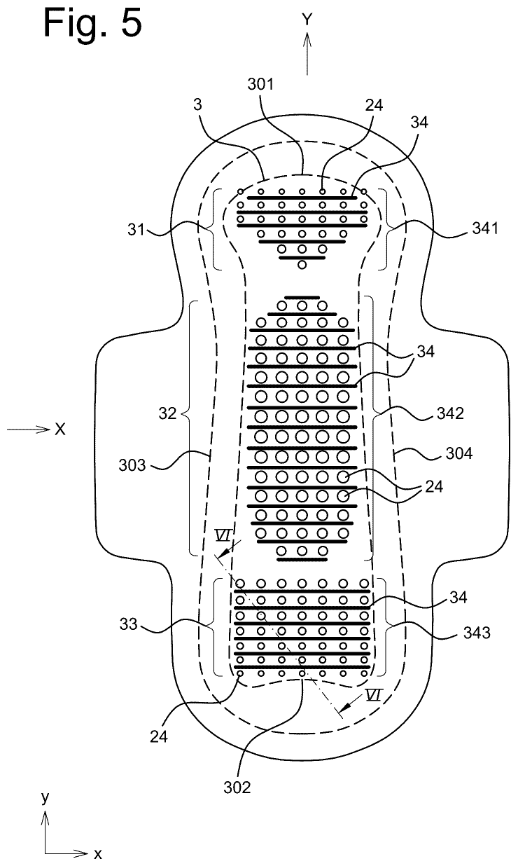

As mentioned above, the provision of the plurality of apertures in the liquid acquisition layer, means that there is less material, which can lead to a decrease in mechanical stability of the absorbent article. This potential decrease can be even further exacerbated when the liquid acquisition layer is also provided with a perforated border line, as the plurality of discrete holes which form the perforated border line means an even further reduction in material. To compensate for this potential decrease in mechanical stability, the absorbent core is provided with a plurality of stabilizing elements. Exemplary stabilizing elements are depicted in FIG. 5.

Turning now to FIG. 5, the absorbent core 3 has a length in the longitudinal direction (y) between a front transverse edge 301 and a rear transverse edge 302. The length comprises a front section 31, a rear section 33, and a central section 32 arranged between the front and rear sections. The absorbent core also has a width in the transverse direction between left side edge 303 and right side edge 304.

The absorbent core comprises a plurality of stabilizing elements 34. Similar to the plurality of apertures of the liquid acquisition layer, the plurality of stabilizing elements may be arranged in one or more subsets. For example, as shown in FIG. 5, the absorbent core comprises three subsets 341, 342, 343 of stabilizing elements, one subset arranged in each of the front, central, and rear sections of the core. In this example, the stabilizing elements comprise a plurality of continuous embossing lines, which are arranged in the transverse direction (x). Orienting the embossing lines in the transverse direction provides stability to the absorbent article in response to pressure imparted upon the article from the legs of a wearer during use.

The embossing lines may vary in length between subsets and/or within a subset. The number of embossing lines per subset may also vary between respective subsets. It should be appreciated that, in order to provide additional mechanical stability to the absorbent article to compensate for the provision of the plurality of apertures and optional perforated border line, the number of stabilizing elements in a subset may be selected relative to the plurality of apertures and optional discrete holes arranged in the portion of the liquid acquisition layer that overlays a respective section of the absorbent core.

As shown in FIG. 5, the first subset 341 has the smallest number of stabilizing elements relative to the other two subsets. The first subset has two lines that are equal or essentially equal in length and three other lines of differing lengths. This number of stabilizing elements, five in this case, corresponds to the arrangement of the plurality of apertures that overlay with the front section of the absorbent core. In this sense, a stabilizing element is arranged between each adjacent row of apertures. This also means that the stabilizing elements can be relatively longer or shorter based on the number of individual apertures in each row. For example, the last stabilizing element in the first subset is arranged between a row of three apertures and a row of a single aperture; the stabilizing element is the shortest of the subset and its length generally corresponds to the length of the row of three apertures. One exception, however, also present in FIG. 5 is the first line which is arranged most proximal to the front transverse edge of the absorbent core. The length of this stabilizing element does not generally correspond to the length of the two adjacent rows of apertures between which it is set; it is shorter. In this case, the first embossing line is shorter in order to avoid overlapping with the edges of the absorbent core.

The second subset 342 comprises the largest number of stabilizing elements relative to the other two subsets. Again, the number of stabilizing elements of the second subset may be selected to correspond to the number of rows of apertures arranged to overlay the central section of the absorbent core. As can be seen in the figure, the majority of the embossing lines in the central section are similarly, if not equally or essentially equally, sized.

Lastly, the third subset 343 comprises yet another configuration of stabilizing elements. Here, the third subset has more embossing lines than the first subset and less embossing lines than the second subset. Additionally, each stabilizing element of the third subset is equally or essentially equally sized with all of the other stabilizing elements within the subset.

In any case, each embossing line is spaced a distance apart from the left and right side edges of the absorbent core. The lines are spaced from the side edges of the core to reduce, if not avoid, the risk of side leakage. In non-limiting embodiments of the present disclosure, the distance at which the stabilizing elements are spaced apart from the edges of the absorbent core may range from 5 to 15 mm, and preferably from 5 to 10 mm.

In addition to the being spaced apart from the side edges of the absorbent core, the stabilizing lines should also be arranged so as to not overlap with either the plurality of apertures or, when provided, the perforated border line. It is desirable to avoid overlap between the stabilizing elements and the apertures/discrete holes because when the stabilizing elements and aperture/discrete holes are stacked one on top of another, this can create a gap or a space between the topsheet and the absorbent core in the assembled article. This gap or space thus prevents the topsheet from contacting the absorbent core in the assembled article.

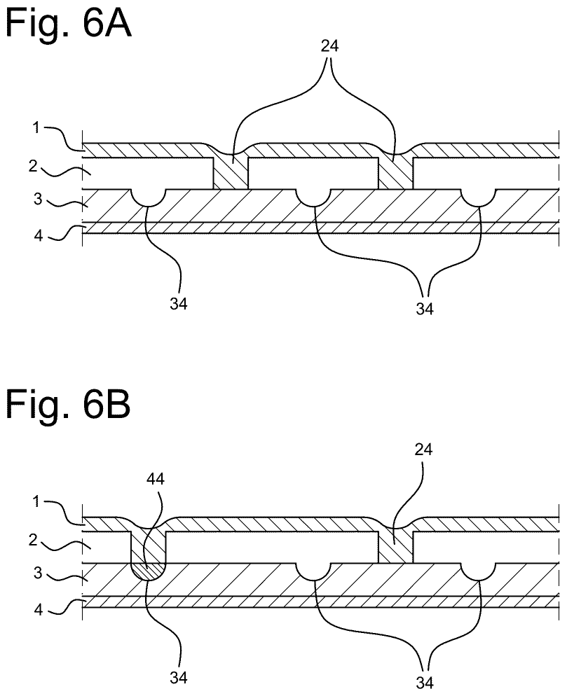

A cross-section of the absorbent core taken along the line VI in FIG. 5 is shown in FIG. 6A. Here, the stabilizing elements 34, which are embossing lines provided in the form of a channel or groove in the absorbent core are arranged directly beneath the liquid acquisition layer. Meanwhile, the apertures 24 formed in the liquid acquisition layer are arranged above portions of the absorbent core that do not contain stabilizing elements. As such, the topsheet may be suitably arranged in the apertures and in direct contact with the absorbent core below.

FIG. 6B shows an alternative cross-section of the absorbent core in which an aperture is arranged directly above a stabilizing element. This arrangement forms a gap or space 44 that is too great for the topsheet to be able to reach the absorbent core.

To facilitate the fit of the absorbent, and by extension, improve the comfort of the article for the wearer during use, the absorbent core may be further provided with one or more shaping lines. Such shaping lines may encourage preferential bending of the article, which in turn, may contribute to improve the fit of the article.

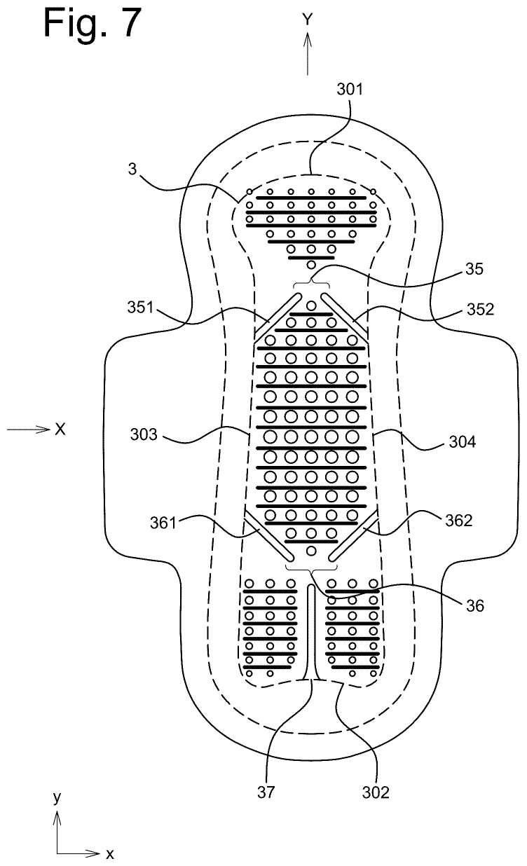

The absorbent core of the absorbent articles described herein, and as shown for example in FIG. 7, may further comprise a first pair of continuous shaping lines 35, a second pair of continuous shaping lines 36 and/or a rear shaping line 37. The first pair of shaping lines may be arranged between the front section and the central section of the absorbent core. The second pair of shaping lines may be arranged between the central section and the rear section. Further, the rear shaping line may be arranged along the central longitudinal axis (Y) in a center of the rear section.

The first pair of continuous shaping lines may comprise a left front line 351 and a right front line 352. The left front line originates from a left side edge in the central section and extends upward at an angle toward the central longitudinal axis. Similarly, the right front line originates from a right side edge in the central section and extends upward at an angle toward the central longitudinal axis. The left front line and right front line do not intersect or overlap with each other, and are thus spaced apart from each other along the central longitudinal axis. This has the advantage of avoiding the formation of a weak spot at which the materials of the core, topsheet, and/or backsheet are more likely to tear, especially during or after manufacture.

The second pair of continuous shaping lines may comprise a left rear line 361 and a right rear line 362. The left rear line originates from the left side edge in the central section and extends downward at an angle toward the central longitudinal axis. Likewise, the right rear line originates from the right side edge in the central section and extends downward at an angle toward the central longitudinal axis. Similar to the first pair of continuous shaping lines, the left rear line and the right rear line do not intersect or overlap with each other and are thus spaced apart from each other along the central longitudinal axis. This arrangement assist to reduce, if not eliminate, the risk of tears or other damages during production and/or assembly of the absorbent article.

As described herein, the continuous shaping lines can provide predetermined or preferential lines along which the absorbent article will fold during use. By providing continuous shaping lines along which the article can fold or deform when compressed during use in a predictable manner, the position of the folds and crease can be controlled in such a manner that the risk of leakage can be minimized. However, the position of the shaping lines should be carefully arranged in order to avoid other properties of the absorbent article from being unduly compromised. For example, the continuous shaping lines should not weaken the structure of the article to a degree that it becomes prone to tearing. Moreover, the shaping lines should not impede the distribution of fluid throughout the absorbent article in such a way that increases the likelihood of leaks.

Further, the positioning of the shaping lines with respect to the absorbent core can be chosen based on the anatomy of the average wearer and adapted based on different user groups or different product applications. The dimensions can also be adapted based on the size, shape, and/or type of article into which the continuous shaping lines are incorporated.

Although all of the exemplary embodiments illustrated in FIGS. 1-5 and 7 depict a disposable absorbent article having wings, it is to be understood that variant(s) of an absorbent article in accordance with the present disclosure can be provided without wings. Moreover, while the above discussion has been exemplified through a sanitary napkin, a hygiene article in accordance with the present disclosure may nevertheless be provided in the form of other absorbent articles such as diapers, incontinence pads, absorbent period underwear, and panty liners.

While the invention has been described herein by reference to certain embodiments, it is to be understood that modifications in addition to those described herein may be made to the structures and techniques described herein without departing from the spirit and scope of the invention. Accordingly, although specific embodiments have been described, they are examples only and are not limiting upon the scope of the invention.

1. A disposable absorbent article having a width in a transverse direction along a transverse axis and a length in a longitudinal direction along a longitudinal axis, the absorbent article comprising:
a liquid-permeable topsheet,
a liquid-impermeable backsheet,
an absorbent core arranged between the topsheet and the backsheet, and
a liquid acquisition layer arranged between the topsheet and the absorbent core, the liquid acquisition layer having a length in the longitudinal direction between a front edge and a rear edge, the length comprising a front portion, a rear portion, and central portion arranged between the front and rear portions, a width in the transverse direction between first and second edges, and a thickness between a top surface and a bottom surface,
wherein the liquid acquisition layer comprises:
a plurality of perforations in the top surface and which do not extend all the way through the bottom surface, and
a plurality of apertures, which extend the thickness of the liquid acquisition layer from the top surface through the bottom surface, and
the absorbent core comprises a plurality of stabilizing elements.

2. The disposable absorbent article of claim 1, wherein the liquid acquisition layer has a multi-layered structure comprising a three-dimensional upper layer which comprises the plurality of perforations, and a fibrous sublayer arranged underneath the upper layer.

3. The disposable absorbent article of claim 1, wherein the plurality of apertures comprises a first subset arranged in the front portion, a second subset arranged in the central portion, and a third subset arranged in the rear portion.

4. The disposable absorbent article of claim 3, wherein the first, second, and third subsets each comprise from 20 to 70% of a total surface area of the respective front, central, and rear portions.

5. The disposable absorbent article of claim 1, wherein the apertures have a diameter of from 1 to 5 mm.

6. The disposable absorbent article of claim 1, wherein the apertures are spaced a distance of from 3 to 30 mm apart from the front edge, the first and second edges, and the rear edge of the liquid acquisition layer.

7. The disposable absorbent article of claim 1, wherein the liquid acquisition layer further comprises a perforated border line around the front, central and rear portions, the perforated border line formed of a plurality of discrete holes, which extend through the thickness of the liquid acquisition layer.

8. The disposable absorbent article of claim 7, wherein the discrete holes are spaced a distance of from 3 to 30 mm apart from the front edge, the first and second edges, and the rear edge of the liquid acquisition layer.

9. The disposable absorbent article of claim 7, wherein adjacent holes of the perforated border line are spaced apart from each other by a distance in a range of from 1 to 8 mm.

10. The disposable absorbent article of claim 7, wherein the discrete holes have a diameter of from 1 to 5 mm.

11. The disposable absorbent article of claim 1, wherein each aperture in the plurality of apertures has a shape selected from one or more of: circle, oval, triangle, square, diamond, or other polygon.

12. The disposable absorbent article of claim 1, wherein the apertures are arranged in rows and/or columns relative to the transverse and longitudinal axes.

13. The disposable absorbent article of claim 1, wherein the apertures of a first subset arranged in the front portion, a second subset arranged in the central portion, and a third subset arranged in the rear portion are arranged in respective patterns, and the respective patterns are preferably different in size and/or shape.

14. The disposable absorbent article of claim 1, wherein the plurality of stabilizing elements comprises a plurality of continuous embossing lines arranged in the transverse direction.

15. The disposable absorbent article of claim 1, wherein the plurality of stabilizing elements are spaced a distance of from 5 to 15 mm apart from left and right side edges of the absorbent core.

16. The disposable absorbent article of claim 1, wherein the plurality of stabilizing elements comprises a first subset arranged in a front section of the absorbent core, a second subset arranged in a central section of the absorbent core, and a third subset arranged in a rear section of the absorbent core.

17. The disposable absorbent article of claim 1, wherein the plurality of stabilizing elements are arranged so as to not overlap with the plurality of apertures or a perforated border line around the front, central and rear portions of the liquid acquisition layer.

18. The disposable article of claim 17, wherein the plurality of stabilizing elements are arranged between adjacent rows of apertures in one or more of a first subset arranged in the first portion, a second subset arranged in the central portion, and a third subset arranged in the rear portion.

19. The disposable absorbent article of claim 1, wherein the absorbent core has a length in the longitudinal direction between a front transverse edge and a rear transverse edge, the length comprising a front section, a rear section, and central section arranged between the front and rear sections, and a width in the transverse direction between left and right side edges, and further comprises a first pair of continuous shaping lines arranged between the front section and the central section, a second pair of continuous shaping lines arranged between the central section and the rear section, and a central rear shaping line arranged along the longitudinal axis in a center of the rear section.

20. The disposable absorbent article of claim 19, wherein the first pair of continuous shaping lines comprise a left front line and a right front line, the left front line originating from the left side edge in the central section and extending upward at an angle toward a central longitudinal axis of the absorbent core, the right front line originating from the right side edge in the central section and extending upward at an angle toward the central longitudinal axis of the absorbent core.

21. The disposable absorbent article of claim 19, wherein the second pair of continuous shaping lines comprise a left rear line and a right rear line, the left rear line originating from the left side edge in the central section and extending downward at an angle toward a central longitudinal axis of the absorbent core, and the right rear line originating from the right side edge in the central section and extending downward at an angle toward the central longitudinal axis of the absorbent core.

22. The disposable absorbent article of claim 1, wherein the length and the width of the liquid acquisition layer are larger than a respective length in the longitudinal direction and a respective width in the transverse direction of the absorbent core such that, when assembled, the liquid acquisition layer overlays the entire absorbent core.

23. The disposable absorbent article of claim 1, wherein the liquid acquisition layer has one of a shape comprising a head part in the front portion, first and second leg members that extend symmetrically about a central longitudinal axis in the central and rear portions, and a transition part arranged between the head part and the first and second leg members, which defines a minimum width in the transverse direction and a rectangular shape.

24. The disposable absorbent article of claim 1, wherein the absorbent core comprises one or more of cellulosic fluff pulp, natural fibers, synthetic fibers, tissue, superabsorbent polymers, absorbent foam materials, absorbent nonwoven materials, and the like.

25. The disposable absorbent article of claim 24, wherein the absorbent core comprises a first absorbent member comprising a nonwoven fabric substrate and a second absorbent member comprising a mixture of pulp fibers and a superabsorbent material.

26. The disposable absorbent article of claim 25, wherein the mixture of pulp fibers and the superabsorbent material form a gradient in which a proportion of the pulp fibers gradually increases from a top surface of the second absorbent member to a bottom surface of the second absorbent member.

27. The disposable absorbent article of claim 1, wherein the topsheet is partially or fully transparent, the liquid acquisition layer comprises a first color, and the absorbent core comprises a second color that is different from the first color.
